(19) United States
(12) Patent Application Publication
(10) Pub. No.: US 2015/0289999 A1
(43) Pub. Date: Oct. 15, 2015

(54) PROSTHESIS SOCKET

(71) Applicant: OTTO BOCK HEALTHCARE GMBH, Duderstadt (DE)

(72) Inventor: Andreas Radspieler, Neubeuern (DE)

(73) Assignee: OTTO BOCK HEALTHCARE GMBH, Duderstadt (DE)

(21) Appl. No.: 14/441,119

(22) PCT Filed: Nov. 15, 2013
(86) PCT No.: PCT/EP2013/003445

§ 371 Date: May 6, 2015

(30) Foreign Application Priority Data
Nov. 15, 2012 (DE) 10 2012 022 414.1

(51) Int. Cl. A61F 2/80

(52) U.S. Cl. CPC A61F 2/80; A61F 2002/805

(57) ABSTRACT

The invention relates to a prosthesis socket having a socket body (2), in which a stump can be inserted, and a flexible liner (5), which, when the prosthesis socket (1) is worn, is located between the socket body (2) and the stump, the liner (5) being firmly connected to the socket body (2) in the proximal region of the latter.

The invention relates to a prosthesis socket having a socket body into which a stump can be inserted, and having a flexible inlay which is arranged between the socket body and the stump when the prosthesis socket is put on, according the preamble of claims 1 and 12.

In the case of arm and leg prostheses, different possibilities for putting the prosthesis on and off are known. In the case of liner systems, the liner is initially turned to the left and pulled over the amputation stump or rolled out onto the stump. The liner, in this case, sits tightly against the stump in such a manner that compression of the stump is reached. In addition, these types of liners can be provided with a fixing pin at their distal end in order to lock the liner to the socket once the stump has been inserted into the socket. The liner sits fixedly on the amputation stump as a result of the gas tightness and frictional adhesion.

A prosthesis socket of this type is known from WO 2009/062489 A1. Over and above this, the prosthesis socket here can also comprise a connection in the socket body to connect a vacuum pump and as a result to increase the vacuum between the socket body and the liner.

Fastening a lanyard, which is pulled through an opening in the socket bottom and can be fixed by means of cable clip, on the distal end of the liner in place of a fixing pin is also additionally known in the case of liner systems. To pull out the prosthesis, the latching mechanism of the locking pin or the cable clip is unlocked and the prosthesis is pulled off the stump.

A disadvantage in the case of the known prostheses, however, is that the putting on and taking off of the prosthesis is only possible by expending a great deal of effort. In order to utilize a prosthesis socket in an optimum manner, it should sit as tightly as possible on the stump. The tighter the stump sits on the socket, the more difficult it is, however, to put on and take off the prosthesis. Over and above this, it can be difficult for patients with limitations with reference to the upper extremities to put on a prosthesis.

The object underlying the invention is to create a prosthesis socket of the type named in the introduction, by way of which putting on and taking off a prosthesis is made easier.

Said object is achieved according to the invention by a prosthesis socket with the features of claims 1 and 12. Advantageous developments of the invention are disclosed in the further claims, the decryption and the figures.

In the case of the prosthesis socket according to the invention, having a socket body into which a stump can be inserted, and having a flexible inlay which is arranged between the socket body and the stump when the prosthesis socket is put on, the inlay is fixedly connected to the socket body in the proximal region of said socket body. The proximal region of the socket body can be the proximal edge of the socket body or the region which connects directly to the proximal edge. The securing of the inlay to the socket body makes it possible for the patient just to have to position the turned-out inlay correctly on the stump and then insert it into the socket body in order to put on the prosthesis or the prosthesis socket. Dividing the donning operation into putting on the inlay or the liner and introducing it into the prosthesis socket is dispensed with, as a result of which in particular patients with additional handicaps find it easier. The same applies to the removal operation which can be made to require less effort by the stump simply being pulled out of the socket body and the inlay rolled off the stump. In addition, the connection between the inlay and the socket body prevents the inlay being released in an unwanted manner from the socket body such that a spatial assignment between inlay and socket body always remains ensured.

A further development of the invention provides that the inlay together with the socket body delimits a fluid pressure chamber which is connected to a device for generating negative pressure or overpressure and is realized so as to be fluid-tight in such a manner that by generating an overpressure or negative pressure in the fluid pressure chamber, a displacement force is exerted in the proximal or distal direction onto the inlay, in particular onto an end region of the inlay. The negative pressure and/or overpressure device can be realized as a pump or compressor, in an advantageous manner the device is realized as a switchable device such that it is able to operate in a first mode as a negative pressure generating device in the form of a vacuum pump and in a second mode as a compressor. In the first mode, stepping into the prosthesis socket can be made easier by the air or the fluid being withdrawn out of the fluid pressure chamber, in the second mode, removal is made easier by the inlay being pressed out of the socket body as a result of the compressed air or the pressurized fluid. In the case of said first embodiment of the prosthesis socket according to the invention, removing the prosthesis socket from the stump is made significantly easier by fluid, in particular compressed air, being introduced into the space between the socket body and the inlay, as a result of which in particular the distal end region of the inlay together with the stump is displaced in the proximal direction relative to the socket body. The stump is consequently pressed out of the socket body by the fluid. At the same time, the pressurized fluid exerts a radially inward force, i.e. directed toward the longitudinal axis of the socket, onto the inlay also from the sides, as a result of which the friction between the socket body and the inlay is reduced and the removal of the prosthesis socket is made additionally easier.

In the case of said removal operation, the inlay, which abuts tightly against the socket body when the prosthesis socket is put on, is inverted “to the left”, as a result of which said inverting operation begins in the distal end region and continues in the proximal direction when the end region of the inlay, initially arranged distally, is displaced in the proximal direction.

In addition, the donning of the prosthesis is also made easier by the prosthesis socket according to the invention. On account of the overpressure generated in the fluid pressure chamber, the opened-out inlay, which is fastened to the socket body, is arranged in a precisely defined manner relative to the socket body and protrudes in a cushion-like manner beyond said socket body in the proximal direction.

In this state the patient can place the stump onto the inlay and displace said inlay in the distal direction until the stump sits together with the inlay correctly in the socket body. In this case, a certain overpressure in the fluid pressure chamber is maintained, as a result of which it is possible to turn the inlay back in a fold-free manner until the entire inlay once again abuts tightly against the socket body. As soon as the inlay abuts against the stump, the first mode can be switched on and the fluid pumped out of the pressure chamber. In principle, it is also possible for an outlet valve to be arranged in the socket body which opens when a predetermined inner pressure in the fluid pressure chamber is exceeded and allows the fluid to flow out when the stump is inserted together with the inlay into the socket body.

The fluid introduced into the fluid pressure chamber is preferably air. However, it is also possible to use other fluids, for example liquids such as water, in place of air.

According to a particularly advantageous further development, the end region of the inlay is realized as a dimensionally stable cap. A cap of this type causes the inversion operation to be effected along the prosthesis socket in a proper and even manner. In addition, as a result of the cap, with the inlay turned out, a shell-like indentation is created for insertion of the stump which enables contact with the distal end face of the stump over the entire area.

In an advantageous manner, the inlay is connected in an air-tight manner to the socket body in the third of the socket body located the farthest proximally. As a result, the inlay can be moved out beyond the socket body in the proximal direction by means of the fluid. As an alternative to this, however, it is also possible to provide the air-tight connection in the middle third or even in the distal third of the prosthesis socket if a correspondingly shorter displacement of the end region of the inlay is sufficient.

In advantageous manner, the air-tight connection between the inlay and the socket body is provided on the inside surface of the socket body. This makes it possible to limit the size of the inlay to a minimum necessary dimension. As an alternative to this, it is also possible to guide the inlay round over the proximal edge region of the socket body and to provide the air-tight connection on the outside surface of the socket body.

In advantageous manner, anti-rotation elements which prevent the inlay twisting relative to the socket body are provided between the inlay and the socket body. These types of anti-rotation elements can include in particular webs and grooves which interact in a positive locking manner and are provided in the longitudinal direction of the socket body on the socket body and on the inlay.

According to a particularly advantageous further development, a feedthrough for a pulling means, e.g. a belt or a pull cord, which is guided through the fluid pressure chamber and is connected to the inlay, in a preferred manner to the distal end of the inlay, is arranged in the distal end region of the socket body. By means of a pulling means of this type, the inlay can be pulled into the socket body together with the stump in a very simple and effortless manner until the stump sits fixedly in the prosthesis socket.

In an advantageous manner, a sealing pulling means feedthrough or pull cord feedthrough which impedes the passage of fluid, is provided in the distal end region of the socket body. As a result, it is not necessary to re-pump an unnecessarily large amount of fluid into or out of the fluid pressure chamber in order to maintain the necessary overpressure or negative pressure in the fluid pressure chamber. A seal can be arranged for this purpose in the feedthrough, as an alternative to this the feedthrough is adapted to the pulling means such that no or only a negligible amount of fluid is able to flow out of or pass into the fluid pressure chamber.

In an advantageous manner the pulling means is connected to a winding means and/or to a force limiting device for limiting the pulling force. A force limiting device of this type provides a safety device which prevents the inlay being pulled too strongly into the socket. The force limiting device can be, for example, an adjustable slipping clutch or a special electronic control means of the winding means. A development of the invention provides that the pulling means is connected to a winding means or a winding device, by means of which the pulling means can be wound up. The winding means can be driven manually or by motor, in the case of a manual development, adaptation to the force level of the patient can be effected by means of a transmission. The motor drive increases the convenience for the patient. The force limiting device can be used with a manual or motor-driven drive for the winding means just as in the case of actuation of the pulling means without any winding means. In the case of actuation without any winding means, the force limiting device can be realized as a slipping handle which is adjustable by means of an adjustable friction factor with reference to the force that can be applied.

The inlay can be secured in a releasable or permanent manner and in a positive locking manner on the socket body. The fastening of the inlay on the socket body can be effected in various ways, for example by means of positive locking, where positive locking elements which correspond to one another are arranged and/or realized on the socket body and the inlay. The positive locking elements can be realized as grooves and thickenings or projections such that to join inlay and socket body, the projections are inserted into the groove or the grooves. This results in a permanent, fixed connection between the inlay and the socket body which, where required, can also be released again, for example in order to replace or to clean the inlay. The mechanical securing as a result of positive locking can bring about a fluid-tight connection such that it is possible to work with negative pressure or overpressure. Fastening means such as pins, seals or other separate components can be used to secure the inlay on the socket body. As an alternative to this or in addition to it, bonding or welding or integral development of the securing means can be undertaken. The inlay can be bonded or welded on the socket body in a circumferential manner or only in portions, where applicable in conjunction with positive locking securing on the socket body. In principle, it is also possible for the prosthesis socket to be produced in one piece, for example using a multiple component injection molding method, such that the socket body and the inlay can comprise the desired characteristics. Permanent fastening does not rule out being able to remove the inlay from the socket body, but ensures assignment between the two components with respect to one another even if the stump has been pulled out of the inlay and removed from the prosthesis socket.

The prosthesis socket according to the invention according to the secondary claim, having a socket body into which a stump can be introduced, and having a flexible inlay which is arranged between the socket body and the stump when the prosthesis socket is put on, provides that the inlay comprises a first inlay wall portion, which is fixed or can be fixed to the socket body, and a second inlay wall portion, between which a fluid pressure chamber is realized which is connected or connectable to a pressure generating device, by means of which a displacement force is exertable in the distal or proximal direction onto the second line wall portion. The inlay can also consist of the two inlay wall portions. The fixing of the inlay on the socket body can be effected in the manner described above, that is in particular by means of a positive locking, non-positive locking or materially-bonding connection or by developing the prosthesis in an integral manner.

In a distally retracted position, the second inlay wall portion can line the first inlay wall portion on the side facing the stump and, together with the first inlay wall portion, can delimit the fluid pressure chamber.

The fluid pressure chamber can be connected to the pressure generating device and realized so as to be fluid-tight in such a manner that by generating an overpressure or negative pressure in the fluid pressure chamber a displacement force is exerted onto the inlay, in particular onto an end region of the second inlay wall portion, in the distal or proximal direction.

Said second embodiment of the prosthesis socket according to the invention differs from the first embodiment in that the fluid pressure chamber is not delimited by the socket body and the inlay, but by two inlay wall portions. The two inlay wall portions, in this case, in a preferred manner form an at least extensively fluid-tight bubble which surrounds the fluid pressure chamber entirely. If the stump is inserted in the prosthesis socket, the two inlay wall portions are packed closely together, the second inlay wall portion lining the first inlay wall portion on the inside surface of said first inlay wall portion. A surface that is closed in relation to the stump which enables pleasant wearing without pressure points being produced on account of discontinuities is provided as a result.

In the case of the second embodiment of the prosthesis socket according to the invention, it is consequently not necessary to provide a fluid-tight seal between the socket body and the inlay. The second embodiment comprises, over and above this, the same advantages which have been described in conjunction with the first embodiment, in particular the securing or fixing of the inlay on the socket body and the assignment of the components with respect to one another obtained as a result.

In addition, it is possible to develop further the second embodiment in the same or quite similar manner as described by way of the first embodiment. In particular, it is possible and advantageous to realize the distal end region of the second inlay wall portion as a dimensionally stable cap, to provide anti-rotation elements between the first and second inlay wall portion and/or to arrange in the distal end region of the socket body a winding means with a pulling means which is guided through the compressed air chamber and is connected to the distal end region of the second inlay wall portion.

In the case of both embodiments, it is expedient, over and above this, when the overpressure generating device comprises a small compressor which is operated electrically in particular and is fastened on the socket body. As an alternative to this, however, it is also possible to use a separate overpressure generating device which is separate from the socket body and is connectable to the fluid pressure chamber by means of a corresponding connection that is present on the prosthesis socket. The same applies to a negative pressure generating device which comprises a vacuum pump and can be fastened on the prosthesis socket. The socket body and/or the inlay can comprise connections for the respective pressure generating device such that they can also be realized so as to be uncouplable and only have to be on the prosthesis socket when required. The pump and the compressor can also be manually driven and realized as one component which can be changed over from suction mode to pressure mode.

The invention is explained in more detail below as an example by way of the drawings, in which:

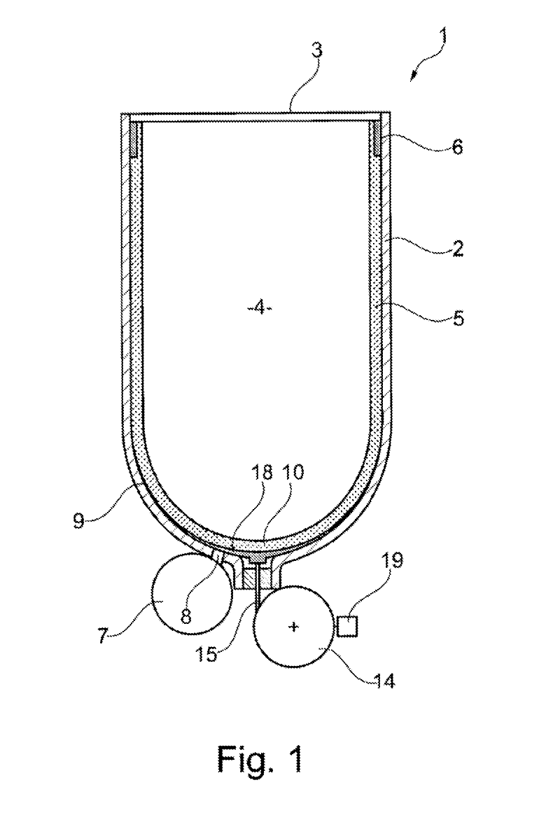

FIG. 1 shows a schematically represented longitudinal section of a first embodiment of the prosthesis socket according to the invention, the inlay being fully retracted into the socket body;

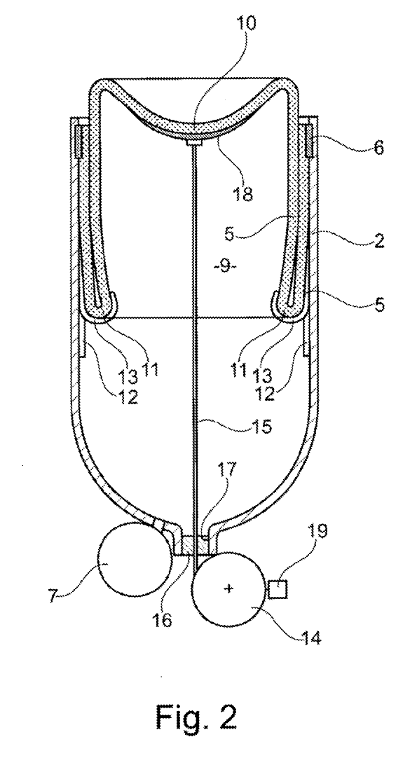

FIG. 2 shows a representation of the prosthesis socket of FIG. 1, the inlay being shown in a semi-turned-out position;

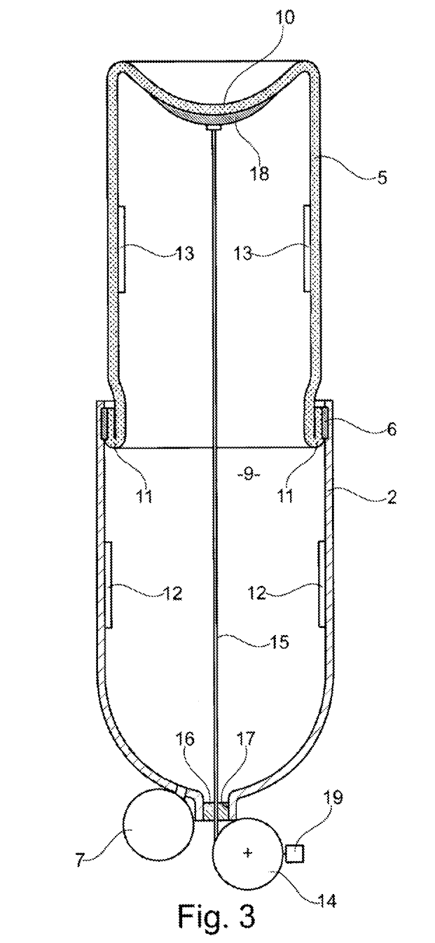

FIG. 3 shows the prosthesis socket of FIG. 1, the inlay being fully turned out;

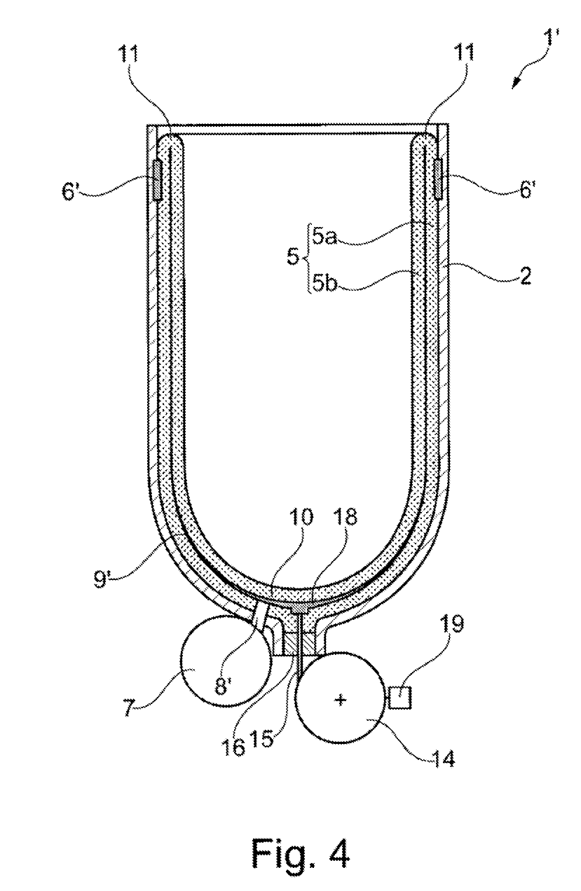

FIG. 4 shows a schematic longitudinal section of a second embodiment of the prosthesis socket, the inlay being fully retracted into the prosthesis socket; and

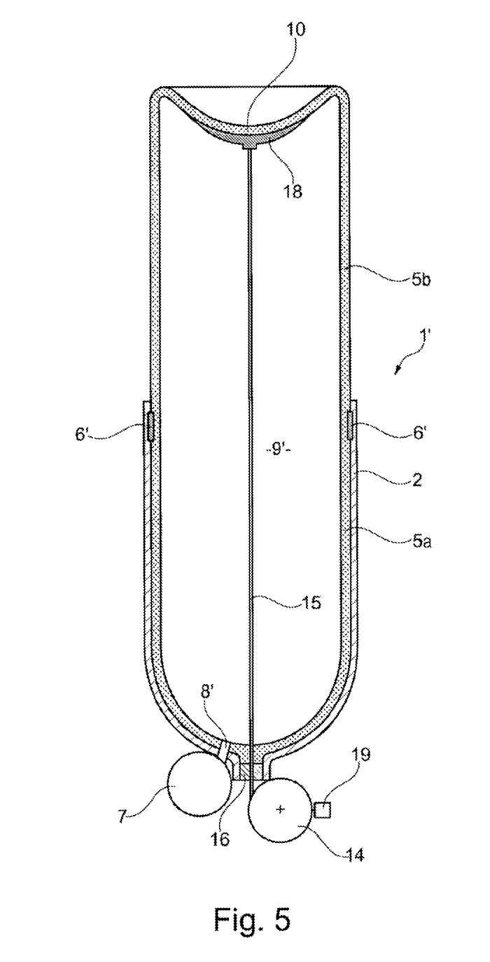

FIG. 5 shows the prosthesis socket of FIG. 4, the inlay being fully turned out.

A first exemplary embodiment of a prosthesis socket 1 according to the invention is explained in more detail below by way of FIGS. 1 to 3. These types of prosthesis sockets 1 are used in particular for arm and leg prostheses and serve for fastening artificial limbs (not shown) to an amputation stump (not shown) of a patient. The artificial limbs, in this case, are fastened in a known manner at the distal end of the prosthesis socket 1.

The prosthesis socket 1 includes a bucket-shaped or bowl-shaped socket body 2 produced from rigid material, in particular from plastics material, metal or a composite material. The socket body 2 comprises a proximal insertion opening 3 for the stump at the proximal end. At its distal end, the socket body 2 is at least extensively closed. The socket body 2 surrounds an interior 4.

The prosthesis socket 1 also includes a flexible inlay 5 produced from an elastic material such as silicone, polyurethane, copolymer or similar materials. In the position of the inlay 5 shown in FIG. 1, which corresponds to that which is assumed with the stump inserted, the inlay 5 covers the socket body 2 in the entire region which would otherwise be contacted by the stump. The inlay 5 consequently serves, on the one hand, as cushioning and, on the other hand, as an elastic intermediate layer which enables force to be transferred over a large area and in a manner that is as free of pressure points as possible between the stump and the socket body 2

The inlay 5 is fastened simply in its proximal end region to a connection 6 which extends in a ring-shaped manner on the socket body 2. In the case of the exemplary embodiment shown, said connection 6 is situated on the inside surface of the socket body 2 and in the third which is situated the farthest proximally, preferably in the proximal end region of the socket body 2. As an alternative to the exemplary embodiment shown, it is also readily possible for the socket body 2 to extend further proximally beyond the connection 6 and also, where applicable, to widen in its diameter in order to create a funnel-shaped insertion opening for the stump.

The connection 6 is realized in such a manner that the gap between the socket body 2 and the inlay 5 is sealed in a fluid-tight manner. In an expedient manner, the connection 6 is an adhesive connection, or, in particular when the inlay 5 is to be developed so as to be removable, as a clamping connection. In the remaining surface regions, the inlay 5 simply abuts loosely against the inner surface of the socket body 2.

In the distal end region of the prosthesis socket 1, there is additionally a pressure generating device 7 which is able to generate both overpressure and negative pressure and is fastened on the socket body 2. Said pressure generating device 7 is connected to the gap between the socket body 2 and the inlay 5 by means of a fluid channel 8 in order, where necessary, to pump fluid into said gap and to generate overpressure therein. Said gap consequently provides a fluid pressure chamber which is designated by way of the reference 9 in FIGS. 1 to 3. In an expedient manner, the fluid is air and the pressure generating device 7 is an electrically operated compressor by way of which compressed air can be pumped into the fluid pressure chamber 9. The compressor can be developed so as to be switchable such that it operates in its second operating mode as a vacuum pump.

As a result of pumping fluid into the fluid pressure chamber 9, a displacement force is exerted in the proximal direction onto a shell-shaped end region 10 of the inlay 5, which in FIG. 1 is arranged distally in the region of the bottom of the socket body 2. This brings about, as can be seen in FIG. 2, a displacement of the end region 10 in the proximal direction relative to the socket body 2. During said displacement, as can be seen clearly in FIGS. 2 and 3, the inlay 5 is inverted, the end region 10 inside the side wall of the inlay 5 moving in the proximal direction in the direction of the insertion opening 3 and then beyond. The inverting operation, in this case, begins, proceeding from the position shown in FIG. 1, in a distal portion of the prosthesis socket 1, a bend edge 11 (FIGS. 2 and 3), which is created by the inlay 5 turning in, being displaced in the proximal direction from said distal portion.

In order to prevent the inlay 5 rotating about the longitudinal axis inside the socket body 2, anti-rotation elements 12, 13, which include webs and grooves, which interact in a positive locking manner, the grooves being provided between the webs, and extend in the longitudinal direction of the prosthesis socket 1, are provided, on the one hand, on the socket body 2 and, on the other hand, on the inlay 5. In an expedient manner, said anti-rotation elements 12 are arranged in the central and/or distal third of the prosthesis socket 1 since the inlay 5 is already prevented from twisting relative to the socket body 1 at the proximal end by the fluid-tight connection 6. As can be seen from FIG. 2, the anti-rotation elements 12 which are provided on the inlay 5 are realized over and above this in a flexible manner in order to enable the inversion of the inlay 5 even in the region of the anti-rotation elements 12.

In order to be able to connect the prosthesis socket 1 in as simple a manner as possible to the stump, a pulling means device or cable pull device is additionally provided which includes a winding means 14 and pulling means 15 which is actuatable by said winding means, e.g. a cable, a strap or a tab. The winding means 14 is fastened on the distal end of the socket body 2 in a manner not shown in any more detail.

As a result of rotating the winding means 14 in different directions of rotation, the pulling means 15 is wound onto the winding means 14 or unwound from it.

The pulling means 15 is guided through a pulling means feedthrough 16 which is situated on the distal end of the socket body 2 in the region of its center axis. The pulling means feedthrough 16 can be, in particular, a plastic plug which is inserted in a sealing manner into an axial opening 17 of the socket body 2. In addition, the pulling means feedthrough 16 comprises a central axial opening, for example a slot or a bore, through which the pulling means 15 is guided and which is sealed in relation to the pulling means 15 in such a manner that the passage of fluid from the fluid pressure chamber 19 to the outside is prevented at least extensively.

Said seal can be effected for example by a seal such as, for example, flexible sealing lips (not shown) which abut against the pulling means 15. In addition, it can already suffice to realize the gap between the pulling means 15 and the axial opening so narrowly that the amount of fluid passing through is negligible.

The pulling means 15 is connected at its proximal end to the inlay 5, in a preferred manner centrally to the end region 10 of the inlay 5. On account of said arrangement, the end region 10, for example proceeding from the expanded position of the inlay 5 shown in FIG. 3, can be pulled in the proximal direction by means of the winding means 14 and consequently pulled into the socket body 2. The inlay 5, in this case, is turned back in the reverse direction until it assumes the position shown in FIG. 1.

So that the end region 10 of the inlay 5 retains its trough-shaped or calotte-shaped form when the inlay 5 is retracted into the socket body 2 and is not deformed into the shape of a cone, the end region 10 is realized as an at least extensively dimensionally stable cap. This can be effected, for example, by a relatively rigid reinforcing layer 18 which is arranged on the side of the end region 10 remote from the stump. Said reinforcing layer 18 forms a shell which supports the end region 10 of the inlay 5 centrally, the diameter of the reinforcing layer 18 being between 50% and 99% of the inside diameter of the inlay 5 in the region of the inlay 5 which adjoins the reinforcing layer 18.

The pulling force of the winding means 14 can be limited by a force limiting device 19 in order to prevent too strong a retraction of the inlay 5 or an overload of the winding means 14 or of the pulling means 15. The winding means 14 can be fastened in an uncouplable manner on the socket body 2.

The method of operation of the prosthesis socket 1 is as follows:

To put on the prosthesis socket 1, first of all the overpressure generating device 7 is activated in order to introduce fluid into the fluid pressure chamber 9. As a result, an overpressure is generated in the fluid pressure chamber 9 which causes the inlay 5 to be opened out (when using compressed air: “blown out”) from the socket body 2 in the manner shown in FIG. 3 into the form of a reverse beaker. The winding means 14, in this case, obtains from an electronic control device the instruction to release the pulling means 15. The end region 10 of the inlay 5 can consequently pull the pulling means 15 in the proximal direction through the fluid pressure chamber 9. The overpressure in the fluid pressure chamber 9 is monitored by an electronic control device and held at a predetermined value.

The patient then places his stump onto the end region of the inlay 5 and presses it lightly in the distal direction. This causes the electronic control device to receive a signal by means of which the winding means 14 is instructed to retract the pulling means 15. Said signal can be effected, for example, by means of a sensor system which monitors the tension of the pulling means 15 and when the stump is placed in position detects the resultant easing of the pulling means 15. The winding means 14 then pulls the end region 10 of the inlay 5 in the distal direction and consequently into the socket body 2, the inlay 5 being turned back into the position shown in FIG. 1 in which it abuts tightly against the socket body 2. In the insertion operation, a certain overpressure is maintained in the fluid pressure chamber 9 so that the inlay 5 is turned back in a defined manner. The fluid displaced out of the fluid pressure chamber 9 during the insertion is drained to the outside by means of an outlet valve provided in the socket body 2. From a certain depth of insertion, for example from approximately 60% of the insertion, a negative pressure can also be generated in the fluid pressure chamber 9 in order to support the inlay 5 abutting tightly against the socket body 2. Said negative pressure can be generated either by switching over the overpressure generating device 7 or by an additional vacuum pump (not shown) which communicates with the fluid pressure chamber 9.

When the inlay 5 is completely inserted, the winding means 14 is locked, as a result of which the inlay 5 and consequently the stump sit fixedly in the socket body 2.

To remove the prosthesis socket 1, proceeding from the position shown in FIG. 1, fluid is pumped into the fluid pressure chamber 9 by means of the overpressure generating device 7 and the winding means 14 is unlocked. The overpressure generated in the fluid pressure chamber 9 brings about a proximal displacement of the end region 10 of the inlay 5, as a result of which the inlay 5 is pressed out of the socket body 2 together with the stump.

A second exemplary embodiment of a prosthesis socket 1 according to the invention is described below by way of FIGS. 4 and 5.

The socket body 2, the overpressure generating device 7 and the pulling means device with the winding means 14 and the pulling means 15 as well as the basic method of operation are realized as in the case of the first embodiment such that reference is made to the above description in this regard.

Different to the first embodiment is that in the case of the second embodiment the inlay 5 is realized in a different manner, the fluid pressure chamber 9′ no longer being arranged between the socket body 2 and the inlay 5, but on its own inside the inlay 5. The result of this is that the connection 6′ between the inlay 5 and the socket body 2 no longer has to be realized in an air-tight manner.

As can be seen from FIGS. 4 and 5, the inlay 5 there is realized from a first inlay wall portion 5a which lines the socket body 2 and can be fixedly connected to said socket body, and a second inlay wall portion 5b which connects to the first inlay wall portion 5a and is preferably integral with said inlay wall portion.

The first inlay wall portion 5a is connected in its proximal end region to the socket body 2 by means of the connection 6′, which can be an adhesive, clamping, positive locking, weld or other bonding connection. As already stated, the connection 6′ does not have to be an air-tight connection. Neither is it necessary for the connection 6′ to extend completely around the inner and outer periphery of the socket body 2. The connection 6′ is arranged in the proximal region of the socket body 2 in the exemplary embodiment shown and provides the transition between the first inlay wall portion 5a and the second inlay wall portion 5b. In principle, it is also possible for the connection 6′ to be arranged further distally or to extend over the entire length of the socket body 2 in order to obtain as large as possible a contact surface. The connection 6′ between the first inlay wall portion 5a and the socket body 2 can also be effected over the entire surface.

The flexible second inlay wall portion 5b is realized in the same or a quite similar manner as the first inlay wall portion 5a and is movable relative to said first inlay wall portion. The second inlay wall portion 5b, as can be seen in FIG. 4, can be retracted into a turned-in position in which it lines the first inlay wall portion 5a on the inside surface of said first inlay wall portion and abuts tightly against said first inlay wall portion. In this state, the inlay 5 is consequently present as a double-walled inlay inside the socket body 2. The bend edge 11, in this case, is situated in direct proximity to the connection 6′. From said position the second inlay wall portion 5b can be turned out into the expanded position shown in FIG. 5. This is effected as a result of fluid being pumped by the overpressure generating device 7 into the fluid pressure chamber 9′ which is situated in the position shown in FIG. 4 between the first inlay wall portion 5a and the second inlay wall position 5b.

The fluid is pumped in by means of a fluid channel 8′ which penetrates both the socket body 2 and the first inlay wall portion 5a and opens out into the fluid pressure chamber 9′.

The pulling means 15, in the case of the second embodiment, is guided in a sealed manner through both the pulling means feedthrough 16 of the socket body 2 and through the first inlay wall portion 5a and is connected to the cap-shaped or calotte-shaped end region 10 of the second inlay wall portion 5b. Said end region 10 is arranged in the position shown in FIG. 4 distally inside the socket body 2 and in the position shown in FIG. 5 proximally outside the socket body 2. The reinforcing region 10 can be realized apart from this in the same manner as in the case of the first embodiment.

If when retracting the second inlay wall portion 5b by means of the pulling means 15, the fluid displaced out of the fluid pressure chamber 9′ is not drained by means of the fluid channel 8′, but by means of a fluid outlet valve which is separate from this, it does not only penetrate the socket body 2, but also the first inlay wall portion 5a in order to open out into the fluid pressure chamber 9′.

With reference to the method of operation, reference is made to the description of the first exemplary embodiment, in the case of the second exemplary embodiment it not being the entire inlay 5 but just the second inlay wall portion 5b which is moved in a corresponding manner. In addition, anti-rotation elements, which can be realized in the same or a similar manner as the anti-rotation elements 12 of the first embodiment, can also be provided between the first inlay wall portion 5a and the second inlay wall portion 5b.

By means of the prosthesis socket 1, 1′ according to the invention, the putting on and taking off of a prosthesis is made considerably easier. Full contact can be readily produced in this case between the distal stump region and the end region 10 of the pressed-out flexible inlay in order to avoid air bubbles and increased buildup of sweat in the socket. In order to avoid the inlay 5 being pressed out and pulled in at an angle, it is expedient when the connection 6, 6′, this means the connecting edge of the inlay 5 in the socket body 2, lies in a plane which is aligned at right angles to the longitudinal axis of the prosthesis socket. The retracting with the winding means 14 can be started either by the touch of a button, flipping a switch, a time delay or, in an expedient manner, by slight pressure onto the end region 10 of the inlay 5. In addition, the overpressure generating device 7 and the winding means 14 are arranged in such a manner on the socket body 2 that the mounting and function of artificial limbs (not shown in the figures) on the socket body 2 is not impeded.

1. A prosthesis socket, comprising:
a socket body into which a stump can be inserted;
a flexible inlay which is arranged between the socket body and the stump when the prosthesis socket is positioned on the stump;
wherein the inlay is fixedly connected to the socket body in a proximal region of the socket body.

2. The prosthesis socket as claimed in claim 1, wherein the inlay together with the socket body defines a fluid pressure chamber which is connected or connectable to a device for generating negative pressure or overpressure in the fluid pressure chamber, the negative pressure or overpressure providing a displacement force that is exerted in a proximal or distal direction onto an end region of the inlay.

3. The prosthesis socket as claimed in claim 2, wherein the end region of the inlay comprises a dimensionally stable cap.

4. The prosthesis socket as claimed in claim 1, wherein the inlay is connected in an air-tight manner to the socket body in a proximal third of the socket body.

5. The prosthesis socket as claimed in claim 4, wherein the air-tight connection between the inlay and the socket body is provided on an inside surface of the socket body.

6. The prosthesis socket as claimed in claim 1, further comprising anti-rotation elements provided between the inlay and the socket body and operable to prevent the inlay from twisting relative to the socket body.

7. The prosthesis socket as claimed in claim 6, wherein the anti-rotation elements include webs and grooves which interact in a positive locking manner and are provided in a longitudinal direction of the socket body on the socket body and on the inlay.

8. The prosthesis socket as claimed in claim 2, further comprising a feedthrough arranged in the distal end region of the socket body, the feedthrough being operable with pulling device that is guided through the fluid pressure chamber and is connected to the inlay.

9. The prosthesis socket as claimed in claim 8, seal which impedes the passage of fluid and is provided in the distal end region of the socket body.

10. The prosthesis socket as claimed in claim 8, wherein the pulling device is connected to at least one of a winding member and a force limiting device for limiting the pulling force.

11. The prosthesis socket as claimed in claim 1, wherein the inlay is secured in a releasable or permanent manner and in a positive locking manner on the socket body.

12. The prosthesis socket, comprising:
a socket body into which a stump can be inserted;
a flexible inlay which is arranged between the socket body and the stump when the prosthesis socket is put on, the inlay comprising:
a first inlay wall portion, which is fixed or can be fixed to the socket body;
a second inlay wall portion;
a fluid pressure chamber positioned between the first and second inlay wall portions, the fluid pressure chamber being connected or connectable to a pressure generating device, wherein a pressure force generated by the pressure generating device provides a displacement force that is exertable onto the second inlay wall portion in a distal or proximal direction.

13. The prosthesis socket as claimed in claim 12, wherein the second inlay wall portion lines the first inlay wall portion on the side facing the stump when the second inlay wall portion is in a distally retracted position.

14. The prosthesis socket as claimed in claim 12, wherein the fluid pressure chamber is fluid-tight in such a manner that by providing an overpressure or negative pressure in the fluid pressure chamber by operation of the pressure generating device, the displacement force is exerted onto an end region of the second inlay wall portion.

15. The prosthesis socket as claimed in claim 14, wherein the end region of the second inlay wall portion comprises a dimensionally stable cap.

16. The prosthesis socket as claimed in claim 12, further comprising anti-rotation elements that are at least one of positioned between the first inlay wall portion and the socket body and positioned between the first and the second inlay wall portion, the anti-rotation elements being operable to prevent at least one of the first inlay wall portion twisting relative to the socket body and the second inlay wall portion twisting relative to the first inlay wall portion.

17. The prosthesis socket as claimed in claim 16, wherein the anti-rotation elements include webs and grooves which interact in a positive locking manner, extend in the longitudinal direction of the prosthesis socket, and are at least one of provided on the socket body and first inlay wall element and provided on the first and second inlay wall portion.

18. The prosthesis socket as claimed in claim 12, further comprising a feedthrough arranged in the distal end region of the socket body, the feedthrough being operable with a pulling device that is guided through the fluid pressure chamber and is connected to the.

19. The prosthesis socket as claimed in claim 18, further comprising a seal which impedes the passage of fluid and is provided in the feedthrough in a distal end region of the first inlay wall portion.

20. The prosthesis socket as claimed in claim 18, wherein the pulling device is connected to at least one of a winding member and a force limiting device for limiting the pulling force.

21. The prosthesis socket as claimed in claim 12, wherein the pressure generating device comprises at least one of an electrically operated compressor and a pump which are fastened on the socket body.
